(19) United States
(12) Patent Application Publication
(10) Pub. No.: US 2014/0373854 A1
(43) Pub. Date: Dec. 25, 2014

(54) METHOD AND STRUCTURE FOR ACHIEVEING ACOUSTICALLY SPECTRUM TUNABLE EARPIECES, PANELS, AND INSERTS

(71) Applicant: John P. Keady, Fairfax Station, VA (US)

(72) Inventor: John P. Keady, Fairfax Station, VA (US)

(21) Appl. No.: 13/859,815

(22) Filed: Apr. 10, 2013

(51) Int. Cl. A61F 11/10

(52) U.S. Cl. CPC A61F 11/10; A61F 2011/085
USPC 128/865

(57) ABSTRACT

At least one exemplary embodiment is directed to an earpiece that includes a fluid in a reservoir where the composition of the fluid or the pressure of the fluid can be modified to vary the insertion loss value of the earpiece.

### CROSS-REFERENCE TO RELATED APPLICATIONS

This application is also a continuation in part of U.S. patent application Ser. No. 13/154,429 filed on 6 Jun. 2011, which claims priority to U.S. patent application No. 61/351,290, filed 4 Jun. 2010, the disclosure of both are incorporated herein by reference in their entirety. This application is also a continuation in part of U.S. patent application Ser. No. 13/609,208 filed on 10 Sep. 2012, which claims priority to U.S. patent application No. 61/532,099, filed 8 Sep. 2011, the disclosure of both are incorporated herein by reference in their entirety. This application is also a continuation in part of U.S. patent application Ser. No. 13/485,466 filed on 31 May 2012, which claims priority to U.S. patent application No. 61/491,447, filed May 31, 2011, the disclosure of both are incorporated herein by reference in their entirety.

### FIELD OF THE INVENTION

The present invention relates to devices that modify acoustic attenuation and reflection, and more particularly, though not exclusively, devices that can be inserted into an ear canal or used as a sound insert or panel.

### BACKGROUND OF THE INVENTION

Hearing protection can take several forms such as earplugs and muffs. Such hearing protection devices attenuate acoustic energy before it reaches the eardrum (tympanum) by creating an insertion loss that is achieved by reflection of the sound waves, dissipation with the device's structure, impedance of the waves through tortuous paths, closing of acoustical valves, and other means. For a hearing protector, the amount of sound pressure level (SPL) reduced, usually measured in decibels (dB), is typically depicted graphically as a function of frequency. Most hearing protection fails to deliver a flat attenuation across frequency spectrum, instead typically providing attenuation which increases in dB as frequency increases; therefore, the attenuation spectrum is typically nonlinear, which affects the perception of sound frequencies across the audible spectrum in different degrees. For this reason, pitch perception and other auditory experiences which rely on frequency-based cues can be compromised by the nonlinear attenuation imparted by conventional hearing protectors. This leads to the need for uniform or “flat” attenuation, which is desirable in many situations, for example, musicians would like to conserve their hearing while hearing an accurate frequency representation of the produced music, or workers who must listen for certain spectral characteristics associated with their machinery or environment. Ferrofluids are composed of nanoscale particles (diameter usually 10 nanometers or less) of magnetite, hematite or some other compound containing iron. This is small enough for thermal agitation to disperse them evenly within a carrier fluid, and for them to contribute to the overall magnetic response of the fluid. This is analogous to the way that the ions in an aqueous paramagnetic salt solution (such as an aqueous solution of copper(II) sulfate or manganese(II) chloride) make the solution paramagnetic.

Particles in ferrofluids are dispersed in a liquid, often using a surfactant, and thus ferrofluids are colloidal suspensions—materials with properties of more than one state of matter. In this case, the two states of matter are the solid metal and liquid it is in. This ability to change phases with the application of a magnetic field allows them to be used as seals, lubricants, and may open up further applications in future nanoelectromechanical systems.

True ferrofluids are stable. This means that the solid particles do not agglomerate or phase separate even in extremely strong magnetic fields. However, the surfactant tends to break down over time (a few years), and eventually the nano-particles will agglomerate, and they will separate out and no longer contribute to the fluid's magnetic response.

The term magnetorheological fluid (MRF) refers to liquids similar to ferrofluids (FF) that solidify in the presence of a magnetic field. Magnetorheological fluids have micrometre scale magnetic particles that are one to three orders of magnitude larger than those of ferrofluids.

However, ferrofluids lose their magnetic properties at sufficiently high temperatures, known as the Curie temperature. The specific temperature required varies depending on the specific compounds used for the nano-particles.

Electrorheological (ER) fluids are suspensions of extremely fine non-conducting particles (up to 50 micrometres diameter) in an electrically insulating fluid. The apparent viscosity of these fluids changes reversibly by an order of up to 100,000 in response to an electric field. For example, a typical ER fluid can go from the consistency of a liquid to that of a gel, and back, with response times on the order of milliseconds. The change in apparent viscosity is dependent on the applied electric field, i.e. the potential divided by the distance between the plates. The change is not a simple change in viscosity, hence these fluids are now known as ER fluids, rather than by the older term Electro Viscous fluids. The effect is better described as an electric field dependent shear yield stress. When activated an ER fluid behaves as a Bingham plastic (a type of viscoelastic material), with a yield point which is determined by the electric field strength. After the yield point is reached, the fluid shears as a fluid, i.e. the incremental shear stress is proportional to the rate of shear (in a Newtonian fluid there is no yield point and stress is directly proportional to shear). Hence the resistance to motion of the fluid can be controlled by adjusting the applied electric field.

One of the current issues with hearing protection and hearing assistance systems is that the attenuation cannot be tuned for a particular situation.

### BRIEF DESCRIPTION OF THE DRAWINGS

Exemplary embodiments of present invention will become more fully understood from the detailed description and the accompanying drawings, wherein:

FIG. 1 illustrates a cartilaginous region and a bony region of an ear canal;

FIG. 2 illustrates general physiology of an ear;

FIG. 3 illustrates a nonlimiting example of an experiment for determining material properties of inflatable elements;

FIG. 4 illustrates the sound pressure levels (SPL) of the upstream microphone (UM) and the downstream microphone (DM) as a function of medium and pressure;

FIG. 5 illustrates the insertion loss (IL) value for three mediums, NaCl, H2O, and Air at 400 mbar gauge pressure;

FIG. 6 illustrates the insertion loss (IL) value for three mediums, NaCl, H2O, and Air at 600 mbar gauge pressure;

FIG. 7 illustrates the insertion loss (IL) value for three mediums, NaCl, H2O, and Air for 400 mbar and 600 mbar gauge pressures;

FIG. 8 illustrates the insertion loss (IL) value for Air for gauge pressures of 350 mbar, 450 mbar, 550 mbar, and 650 mbar gauge pressures;

FIG. 9 illustrates the insertion loss (IL) value for H2O for gauge pressures of 350 mbar, 450 mbar, 550 mbar, and 600 mbar gauge pressures;

FIG. 10 illustrates the insertion loss (IL) value for two mediums, H2O, and Air for 450 mbar and 550 mbar gauge pressures;

FIG. 11 illustrates a general mathematical model of an earplug using a membrane;

FIG. 12 illustrates an experimental test system that can be used to test attenuation and reflection characteristics both in a subject and for panel design;

FIGS. 13-18 illustrate non-limiting examples of earplugs with modifiable attenuation;

FIG. 19 illustrates a detachable earplug pumping system in accordance with at least one exemplary embodiment; and

FIG. 20 illustrates a lanyard earplug system in accordance with at least one exemplary embodiment.

FIGS. 21A-23 are schematic diagrams illustrating non-limiting examples of earplugs with modifiable attenuation.

FIG. 24 is a schematic diagram illustrating a detachable earplug pumping system in accordance with at least one exemplary embodiment.

FIGS. 25A-C are schematic diagrams illustrating a lanyard earplug system in accordance with at least one exemplary embodiment.

FIG. 26 is a schematic diagram illustrating a hearing protection device embodiment of the invention.

FIG. 27 illustrates an acoustic shaping panel in accordance with at least one exemplary embodiment.

FIG. 28 illustrates a cross section of the panel illustrated in FIG. 27.

FIG. 29 illustrates attachment of the panels of FIG. 27 on a wall in accordance with at least one exemplary embodiment.

FIG. 30A illustrates cross section of an acoustic shaping panel in accordance with at least one exemplary embodiment.

FIG. 30B illustrates a close-up of the medium illustrated in FIG. 30A.

FIGS. 31A, 31B, 31C, and 31D illustrate variations of cross sections of acoustic shaping panels in accordance with various exemplary embodiments.

FIGS. 32A, 32B, and 32C illustrate the configuration and operation of at least one exemplary embodiment.

FIG. 33 illustrates an earplug in accordance with one exemplary embodiment.

FIGS. 34A, 34B, and 34C illustrate the configuration and operation of at least one exemplary embodiment.

FIGS. 35A, 35B, 35C, and 36 illustrate the configuration and operation of at least one exemplary embodiment.

FIGS. 37 and 38 illustrate a helmet with a liner in accordance with at least one exemplary embodiment.

FIGS. 39-40 illustrates various flexible distal ends developed, while

FIG. 41 illustrates a novel distal end spiral feed system which enhances uniform expansion about a stent.

FIGS. 42-44J illustrates multiple examples of embodiments for earplugs, hearing aids, and earpieces.

### DETAILED DESCRIPTION OF EXEMPLARY EMBODIMENTS

The following description of exemplary embodiment(s) is merely illustrative in nature and is in no way intended to limit the invention, its application, or uses.

Exemplary embodiments are directed to or can be operatively used on various passive earplugs for hearing protection or electronic wired or wireless earpiece devices (e.g., hearing aids, ear monitors, earbuds, headphones, ear terminal, behind the ear devices or other acoustic devices as known by one of ordinary skill, and equivalents). For example, the earpieces can be without transducers (for a noise attenuation application in a hearing protective earplug) or one or more transducers (e.g. ambient sound microphone (ASM), ear canal microphone (ECM), ear canal receiver (ECR)) for monitoring/providing sound. In all of the examples illustrated and discussed herein, any specific values should be interpreted to be illustrative only and non-limiting. Thus, other examples of the exemplary embodiments could have different values.

Processes, techniques, apparatus, and materials as known by one of ordinary skill in the art may not be discussed in detail but are intended to be part of the enabling description where appropriate. For example specific materials may not be listed for achieving each of the targeted properties discussed, however one of ordinary skill would be able, without undo experimentation, to determine the materials needed given the enabling disclosure herein.

Notice that similar reference numerals and letters refer to similar items in the following figures, and thus once an item is defined in one figure, it may not be discussed or further defined in the following figures. Processes, techniques, apparatus, and materials as known by one of ordinary skill in the relevant art may not be discussed in detail but are intended to be part of the enabling description where appropriate.

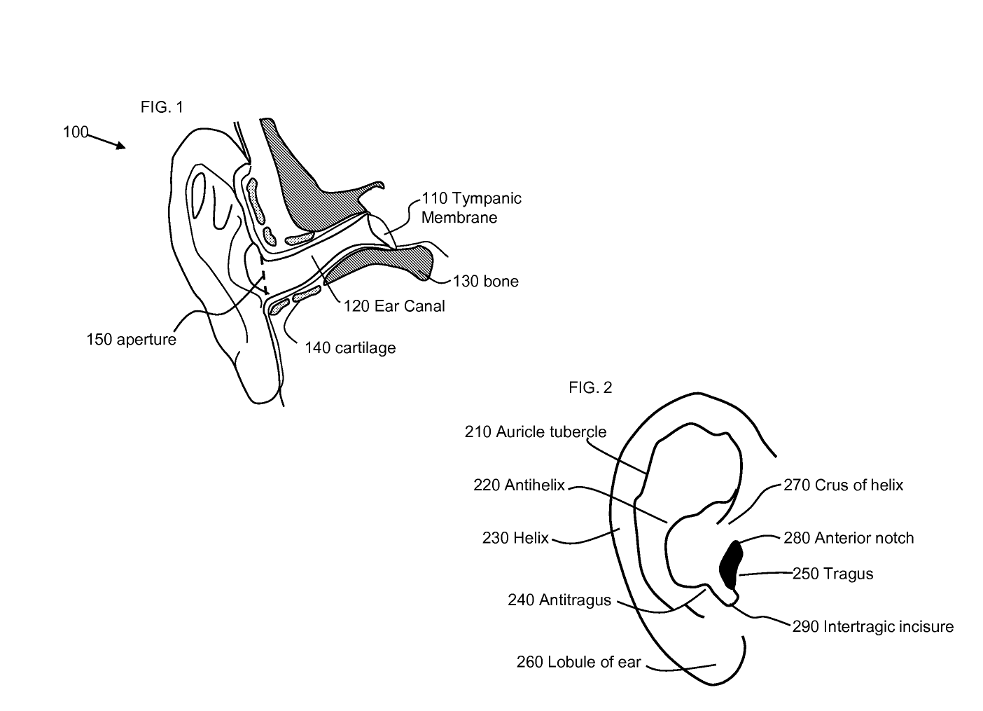

FIG. 1 illustrates a generic cross section of an ear canal 100, including a cartilaginous region 140 and a bony region 130 of an ear canal 120. The entrance of the ear canal 120 is referred to as the aperture 150 and defines a first end of the ear canal while the tympanic membrane 110 defines the other end of the ear canal 120.

FIG. 2 illustrates general outer physiology of an ear, which includes a, auricle tubercle 210, the antihelix 220, the helix 230, the antitragus 240, tragus 250, lobule of ear 260, crus of helix 270, anterior notch 280, and intertragic incisures 290.

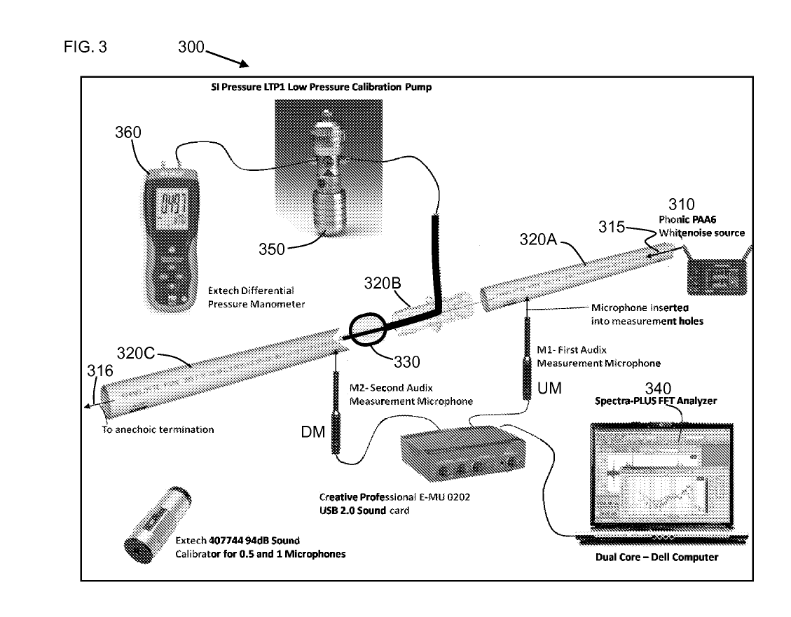

FIG. 3 illustrates a nonlimiting example of an experiment for determining material properties of inflatable elements. To isolate the variations in ear canal lengths, ear canal cross sections and insertion depths of earpieces (e.g., earplugs, in-the-canal hearing aids) an experimental setup 300 was constructed as illustrated in FIG. 3. A noise source 310 (e.g., Phonic PAA6) generates acoustic source waves 315 (e.g., pink noise, white noise) which travel down an acoustic tube 320A where the incident acoustic signal is measured by an upstream first microphone (e.g., M1 or UM, Audix Measurement Microphone). The test sample 330 (e.g., balloon, isolated chamber) can be filled with various fluids (e.g., air, water, water with agents) and inserted into a portion 320B of the tunnel such that the acoustic source waves impinge one side of the test sample, travels through the test sample, and exit the opposite or adjacent (not shown) side of the test sample, where a downstream microphone (e.g., M2 or DM) measures the exiting acoustic waves. To minimize reflections from the end of the downstream tube the system is set to have an anechoically terminated end, which is accomplished by length (>75 ft) so as to gradually diminish the energy of the travelling wave 316 via wall interaction, and by having small strands of string near the end to absorb more of the energy in the wave. The data from the two microphones M1 and M2 are obtained to extract acoustical spectrum information (e.g., using FFT analyzer software such as 340 Spectra-PLUS™ FFT Analyzer). For example, when measuring insertion loss (IL), measurements are taken with M2 prior to insertion of a test sample, then a test sample inserted and measurements retaken with M2. Using the same sound source in both measurements, the difference in the two measurements is defined as insertion loss (IL). For discussion herein with regards to tunnel data IL is approximated when using balloons by a difference in the uninflated M2 measurements (i.e. pressure of 000 mbar gauge pressure) and an inflated M2 measurement. The pressure of a test sample is varied by use of a pressure pump 350 (e.g., SI Pressure LTP1™ Low Pressure Calibration Pump), and monitored by reading the pressure from a pressure gauge 360 (e.g., Extech™ Differential Pressure Manometer).

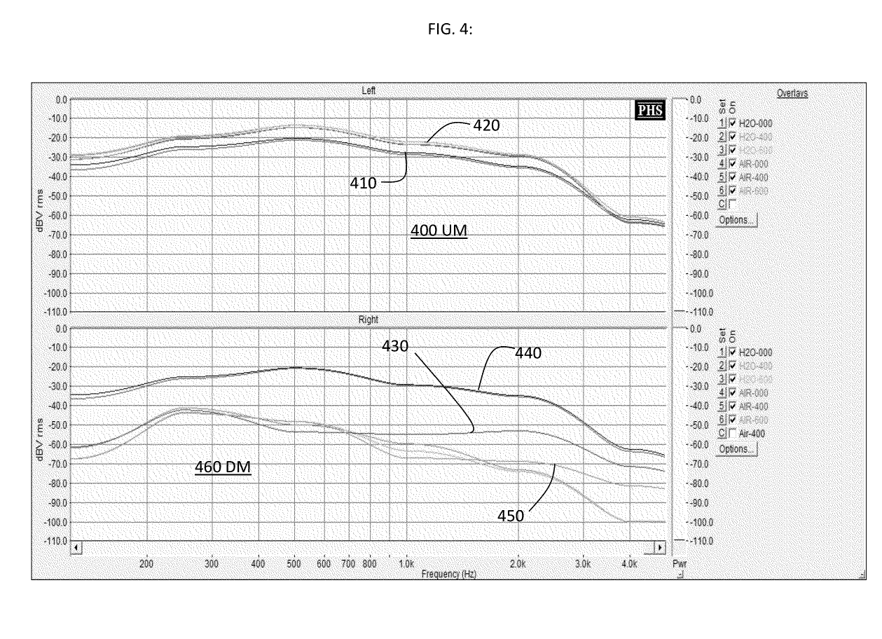

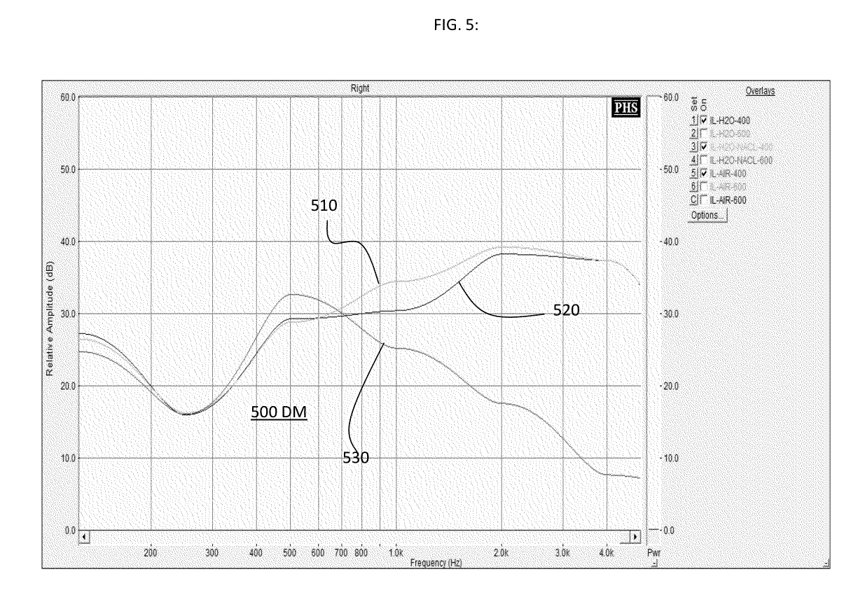

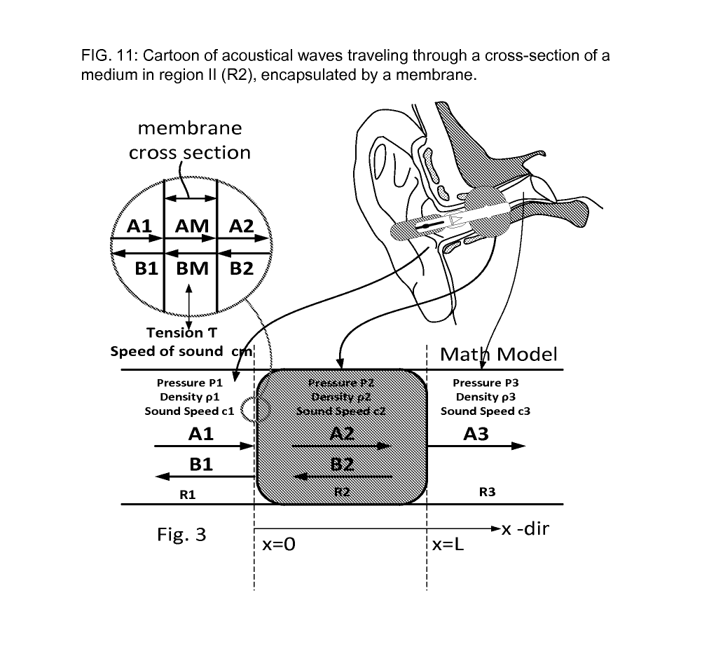

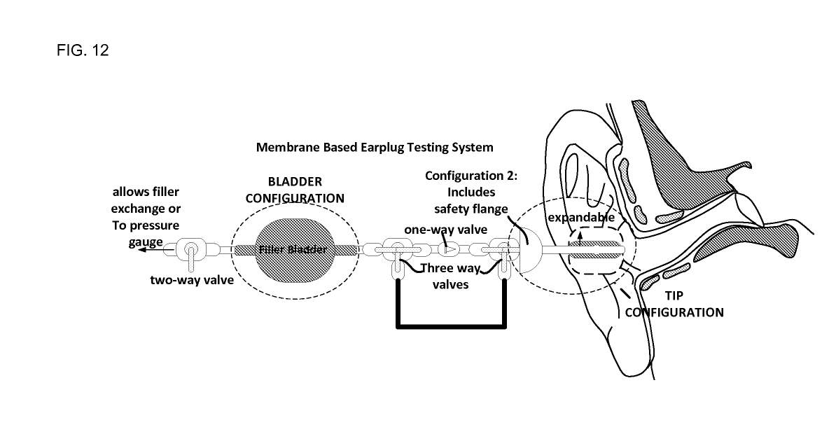

FIG. 4 illustrates the sound pressure levels (SPL) of the upstream microphone (UM) and the downstream microphone (DM) as a function of medium and pressure. dB Values rms between water and air at 000 mbar, 400 mbar, and 600 mbar gauge pressure are illustrated. A larger value indicates higher SPL values, thus a value of −10 dB is an increase of 20 dB in SPL value from −30 dB. Note that the values for 000 mbar represent the uninflated value and the insertion loss (IL) can be obtained by subtracting the 000 mbar value from the pressure values for the downstream microphone (DM). IL values are presented on the next plot (FIG. 5); note also that the plotting values are 1-octave values and hence have been averaged from the narrowband data, thus details in the narrow band data are lost. However the 1-octave values allow more direct comparison to human subject data (FIGS. 11 and 12).

The top panel illustrates upstream microphone 400 (UM) measurements under six conditions, water as the medium under three pressures: 000 mbar (blue), 400 mbar (green), and 600 mbar (light blue); and air as the medium under the same three pressures: 000 mbar (light purple), 400 mbar (red), and 600 mbar (orange). Note that the pressure conditions separate into two general separate lines, the first with no inflation for example 410, and a second line where the two non-zero pressure values generally overlap into a single line 420. Thus generally independent of pressure in the sample, an increase of about 7 dB is measured upstream of the test sample. One possible interpretation is that 7 dB of incident energy is reflected from the interface.

The bottom panel illustrates downstream microphone 460 (DM) measurements under six conditions, water as the medium under three pressures: 000 mbar (blue), 400 mbar (green), and 600 mbar (light blue); and air as the medium under the same three pressures: 000 mbar (light purple), 400 mbar (red), and 600 mbar (orange). Note that the pressure conditions separate into two general regions, the first region is associated with no inflation 440 where irrespective of medium, as one might expect, the lines overlap. The other region varies depending upon medium and pressure. For example, a red line marks dB values for air at 440 mbar and the orange line dB values for 600 mbar. In general as the pressure increases the rms dB value decreases in value as measured by DM. Note that between a frequency of 300-700 Hz an increase in pressure is not associated with an decrease measured value at DM. Note that both UM and DM measurements have roughly a frequency independent standard deviation of <0.2 dB.

FIG. 5 illustrates the insertion loss (IL) values 500 for three mediums, NaCl, H2O, and Air at 400 mbar gauge pressure as measured by the downstream microphone DM. Note that a larger IL value is associated with more energy being removed from the initial acoustic wave by the test sample. As illustrated the three different mediums, distilled H₂O with 1.95 mg/L NaCl (light blue line) 510, distilled H₂O (blue) 520, and Air (red) 530, are distinguishable. For example air provides less IL after 700 Hz than H₂O 520 and H₂O+NaCl mixture 510. Note that H₂O 520 and H₂O+NaCl mixture 510 have similar profiles below 700 Hz and above 3 kHz. Between 700 Hz-3 KHz the IL values 510 and 520 differ such that an H₂O+NaCl mixture provides more IL. Note that although an H₂O+NaCl mixture is illustrated, other mixtures (e.g., with sucrose, alcohol, mineral oil) can be used to tailor specific increases or decreases in IL as a function of frequency for a given pressure.

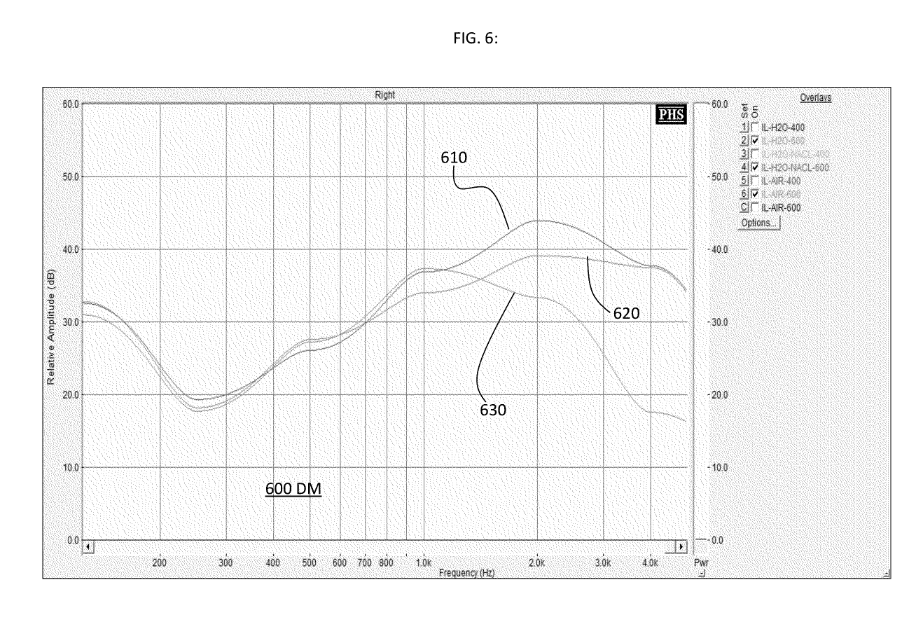

FIG. 6 illustrates the insertion loss (IL) values 600 for three mediums, NaCl, H2O, and Air at 600 mbar gauge pressure as measured by the downstream microphone DM. Note that a larger IL value is associated with more energy being removed from the initial acoustic wave by the test sample. As illustrated the three different mediums distilled H₂O with 1.95 mg/L NaCl (light blue line) 610, distilled H₂O (blue) 620, and Air (red) 630 are distinguishable. For example air provides less IL after about 1.5 kHz than H₂O 620 and H₂O+NaCl mixture 610. Note that the decrease with air as a medium after 1.5 kHz differs from the 400 mbar value of 700 Hz. Thus at increased pressures air 630 provides less IL than H₂O 620 and H₂O+NaCl mixture 610 above a higher frequency. Thus generally as the test sample pressure is increased, the IL profiles also vary, facilitating using controllable pressure values to obtain design IL profiles. For example, if an earplug uses air and an IL value above 700 Hz in unimportant for the particular use, then an earplug can be designed to have an internal balloon pressure of about 400 mbar, whereas if the IL value above 1.5 kHz is unimportant then the earplug balloon can be designed to have an internal pressure of 600 mbar.

Note that H₂O 620 (green) and H₂O+NaCl mixture 610 (red) have similar profiles up to about 700 Hz. Above 700 Hz, the IL values 610 and 620 differ such that an H₂O+NaCl mixture provides more IL. Note that although an H₂O+NaCl mixture is illustrated, other mixtures (e.g., with sucrose, alcohol, mineral oil) can be used to tailor specific increases or decreases in IL as a function of frequency for a given pressure. Thus, if an earplug is designed for use with distilled water, the IL value can be varied at different frequencies by adding agents (e.g., NaCl). If one wishes to increase the IL above 700 Hz one could add a mixture of NaCl and distilled water 620.

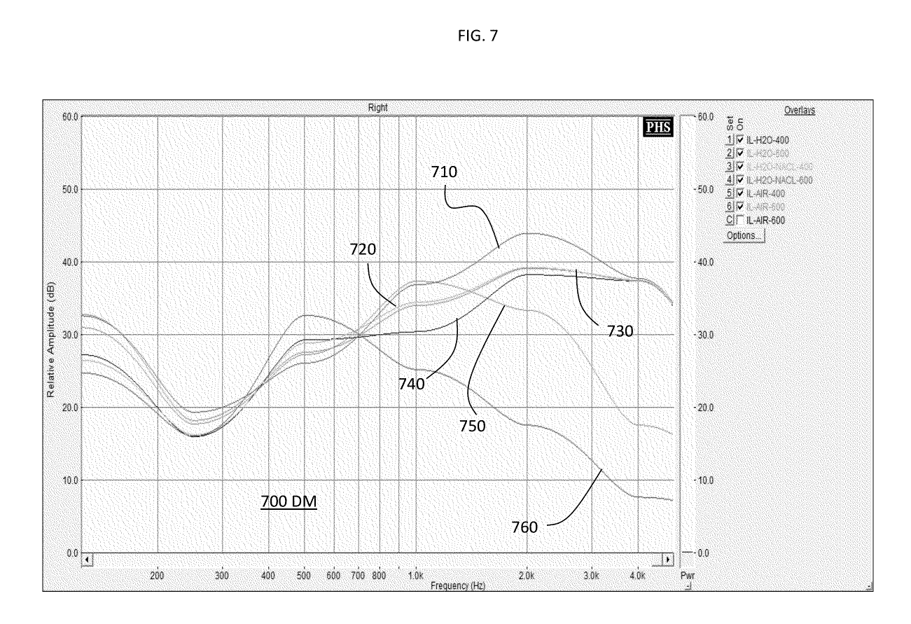

FIG. 7 illustrates the insertion loss (IL) value 700 for three mediums, NaCl, H2O, and Air for two pressures 400 mbar and 600 mbar gauge pressures as illustrated in FIGS. 5 and 6 for ease of comparison.

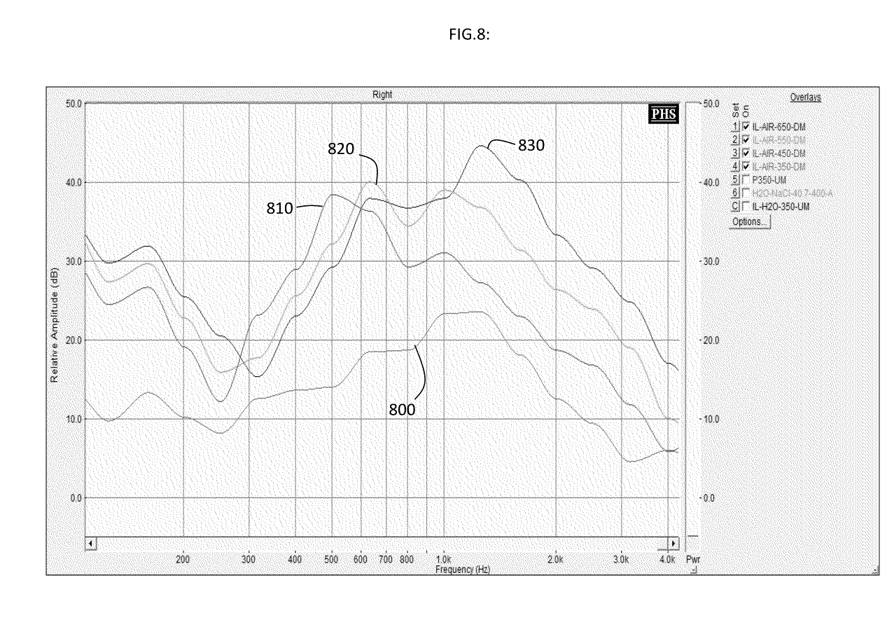

FIG. 8 illustrates the insertion loss (IL) value for Air for gauge pressures of 350 mbar (800), 450 mbar (810), 550 mbar (820), and 650 mbar (830) gauge pressures. In general as the pressure of a test sample increases the IL value increases for frequencies less than about 300 Hz and greater than about 1 kHz. Between about 300 Hz and 1 kHz the pressure with the larger IL depends upon frequency. For example, a pressure of 450 mbar has a larger IL value than other pressures at about 500 Hz, while a pressure of 550 mbar has the largest IL value at about 650 Hz. Thus pressure can be varied in an earplug device to modify the frequency at which the greatest IL is provided. For example, suppose the frequency of an offending noise source gradually increases in frequency. An air-filled earplug with interactive pressure control could increase the pressure of an earplug balloon to maintain suppression of the noise source as its frequency increased.

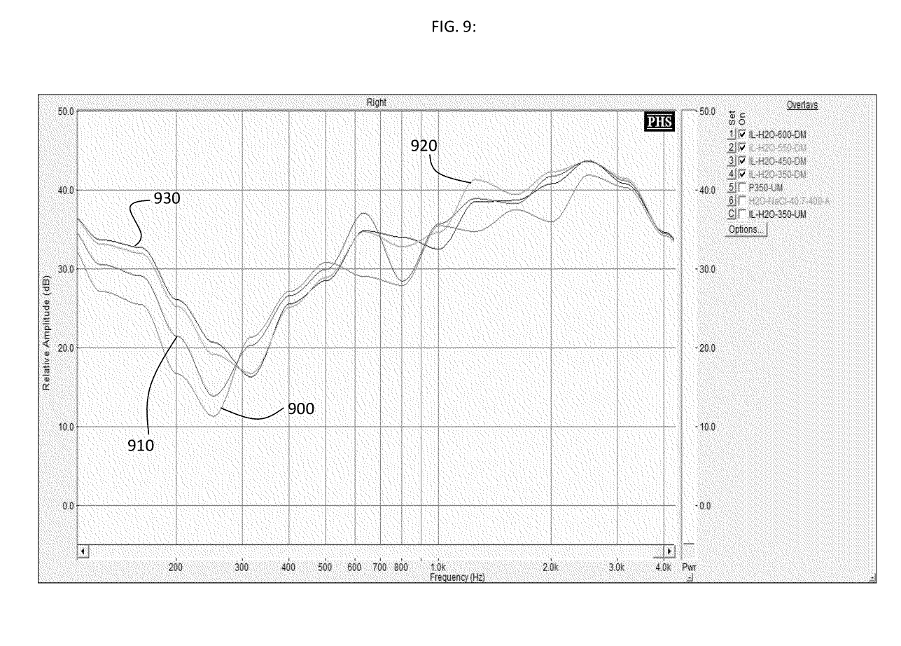

FIG. 9 illustrates the insertion loss (IL) value for H₂O for gauge pressures of 350 mbar (900), 450 mbar (910), 550 mbar (920), and 600 mbar (930) gauge pressures. In general as the pressure of a test sample increases the IL value increases for frequencies less than about 300 Hz. Above about 300 Hz the pressure with the larger IL depends upon frequency. For example a pressure of 450 mbar has a larger IL value than other pressures at about 625 Hz, while a pressure of 550 mbar has the largest IL value at about 1.25 kHz. Thus pressure can be varied in an earplug device to modify the frequency at which the greatest IL is provided. For example, suppose a flatter frequency dependent IL is desired between frequencies of about 500 Hz and 800 Hz, then the pressure of an H₂O filled earplug bladder can be set to about 350 mbar and if an increase of IL is needed within this range then the pressure can be increased.

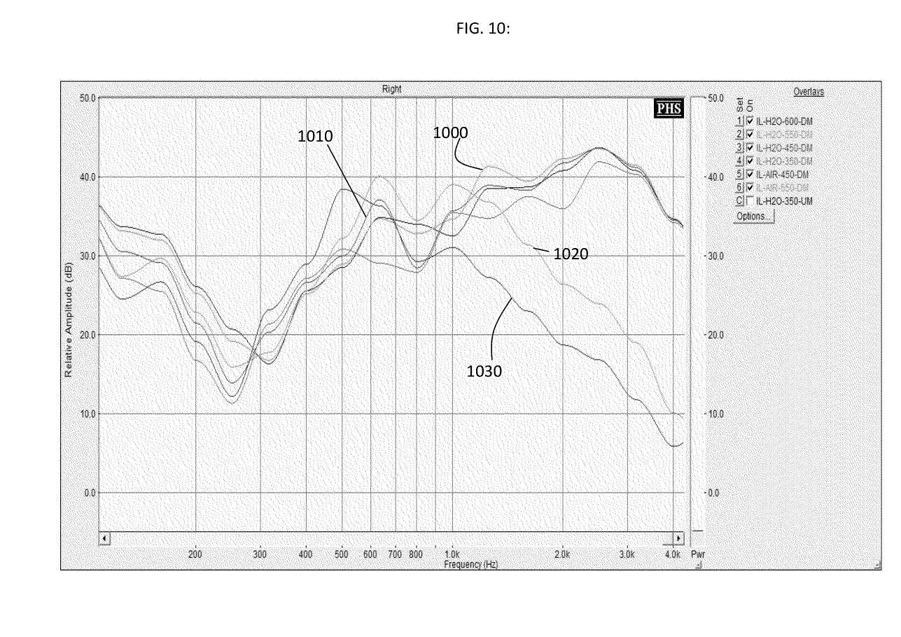

FIG. 10 illustrates the insertion loss (IL) value for the H₂O values of FIG. 9 and two air values for comparison Air at 450 mbar (1030) and 550 mbar (1020) gauge pressures. Note that peak IL values differ from the fluid used (e.g., air or H₂O). For example, if an earplug device is designed to maximize IL at 500 Hz, then one can use air at 450 mbar, where if one wishes to maximize the IL at about 650 Hz the air pressure can be increased to 550 mbar. If one wishes to design an earplug to maximize IL at about 1.25 kHz then one can use H₂O at a pressure of about 550 mbar. Note that a flatter IL profile when using H₂O can be obtain between frequencies about 500 Hz and 1 kHz by setting the H₂O pressure to about 550 mbar as opposed to 450 mbar.

The extent of the earplug can be modeled as a region extending from x=0 to x=L with an incident pressure wave A1 (FIG. 11). The reflectance of the pressure wave and transmission of the pressure wave will depend upon the impedance (Z=ρc) between two regions. The membrane itself can also be considered a region separating region 1 and region 2. Between two regions the Reflection (R) coefficient and Transmission (T) coefficient can be derived using interface boundary conditions BC1 (continuity of pressure) and BC2 (continuity of particle velocity).

A1−B1=A2+B2 (continuity of pressure) (1)

A1−B1=(Z1/Z2)(A2−B2) (continuity of particle velocity) (2)

Note that equations (1) and (2) are generally used across any boundary between two regions. If we treat the membrane as the second region we will get the relationships:

A1+B1=AM+BM (continuity of pressure) (3)

A1−B1=(Z1/ZM)(AM−BM) (continuity of particle velocity) (4)

For a membrane the speed of sound in the membrane, (cm), is a function of the tension force per unit length (Tₗ) and the surface density (m, mass per unit area), and can be expressed as:

cm=\sqrt{Tₗ/m} (5)

Thus ZM can be expressed as ZM=ρₘ\sqrt{Tₗ/m}, whereas Z1=ρ1*c1=(1 Kg/m³)(343 m/sec, in air)=343, and using roughly ρₘ=1100 Kg/m³ (for rubber) and a tension of about Tₗ=(1.2 atm*101300 N/m²)*(π)*(0.005 m)²/0.01 m≈954 N/m, and m=(1100 Kg/m³)*(0.0001 m)/[(π)*(0.005 m)²]≈1401 Kg/m² one can obtain about ZM≈907, . . . so that roughly the ratio Z1/ZM=0.38. Note that for a membrane earplug the filler pressure can be varied and hence the tension force can be varied. Note that a simple examination of continuity of particle velocity (2) results in:

A1−B1≈(0.38)(AM−BM) (continuity of particle velocity) (6)

Thus reflectivity increases at the membrane interface (essentially B1 approaches A1). The unique aspect of membrane earplugs is that the tension can be varied by increasing the pressure in the bladder and the relative speeds of sound can be varied by changing the filler fluid. If one uses a filler fluid of water H2O as a comparison to the aforementioned air, Z2=(1500 m/sec)(1000 Kg/m³)=1500000. In a more general analysis the Reflectivity coefficient (R), examining only the air-filler interface, can be reduced, for when k2L<<1 (a small membrane thickness), as:

R=B1/A1≈[(Z2−Z1)/(Z2+Z1)]≈1499657/1500343=0.9995 (7)

This shows a large reflection coefficient, when the filler is H2O. Note that the value of Z2 is determined by the filler fluid medium and can be tailored depending upon desired attenuation performance.

At least one exemplary embodiment of the present invention employs a simple stretch membrane (i.e., “balloon”) approach, wherein an inflatable, lightweight balloon is inserted into the ear canal in its deflated state, and then inflated once inside the canal. This insertion configuration affords its own additional advantages in the realm of having an in-ear product that is undersize compared to the diameter of the ear canal prior to insertion, and then expands once inside the canal, unlike most other earplug products on the market, including the Ety High Fidelity™ earplug, which are sized to be oversize the ear canal prior to insertion, and thus require squeezing or compression upon insertion, making insertion more difficult.

FIG. 12 illustrates a method for testing various configurations. The membrane-based earplug testing system comprises in general a tip configuration and a bladder configuration. The bladder configuration includes a filler bladder where the filler bladder is a medical balloon that is pre-shaped but deformable. The tip configuration includes a compliant tip that is an expandable elastic medical balloon that conforms to a stent. The stent connects the filler bladder to the compliant tip. The filler bladder can be deformed forcing filler material to the compliant tip which expands to fill an ear canal. The material is kept from flowing back to the filler bladder by a deformable one way valve. The deformable one-way valve (made of compliant rubber-like material) can be deformed by a user to allow back flow to the bladder. The system additionally includes two way and three way valves for the relief of pressure, filler exchange, and pressure measurement. The one way valve, two way valve, and three way valves are valves that can be included in housing, with attached medical luer locks that can then be fitted to the stent. In one of the test configurations the tip configuration includes a safety flange to determine whether inclusion of a safety flange affects localization. Likewise the bladder configuration includes a medical pre-shaped balloon of about 1 cc volume attached to luer lock connectors at either end. Various fillers can be used for example H2O, H2O+NaCl, H2O+Alcohol, Alcohol, MR fluid, and Air, note that this list is a non-limiting example only.

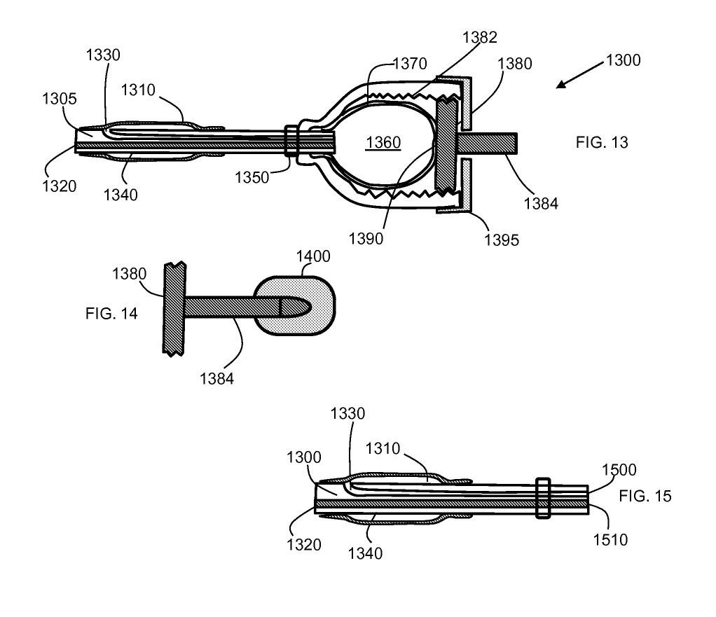

FIGS. 13-18 illustrate non-limiting examples of earplugs with modifiable attenuation. FIG. 13 illustrates an earpiece (e.g., earplug, headphone, hearing aid) that includes a first reservoir 1310 (e.g., Urethane balloon, silicon balloon) fed by a channel (tube) 1330 in a stent 1300. The stent 1300 can be fabricated from various materials (e.g., silicon, urethane, rubber) and can include internal channel (tubes), for example tubes 1330 and 1320. The stent can also be a multi-lumen (i.e., multi-passageway) stent where the channels/tubes are various lumens of the multi-lumen stent. The first reservoir 1310 can be connected to a second reservoir 1370 via the tube 1330. Thus a fluid 1360 can be transferred between the first reservoir 1310 and the second reservoir 1370 by pressing against the second reservoir 1370 or by pressing against the first reservoir 1310. Additionally the reservoirs (1370 and 1310) can be fabricated from stressed membranes (e.g., silicone) so that when fluid is inserted into the reservoirs a restoring force presses against the fluid 1360 by the membrane. For example if the second reservoir was fabricated from a compliant balloon with an initial state of collapse, then filling the second reservoir 1370 with fluid 1360 would stretch the membrane such that the membrane would seek to press against the fluid 1360. If the first reservoir restoring force caused by its membrane is less than that of the second reservoir 1370 then the fluid 1360 will move via tube 1330 into the first reservoir. Alternatively a structure can press against the second reservoir 1370 pushing against the fluid 1360 moving a portion of the fluid in to the first reservoir. FIG. 13 illustrates a non-limiting example of a structure that includes a piston head 1380, a front surface of the piston head 1390 connected to a stem 1384. The structure can lie within a housing that has optional internal threads 1382, which optional threads on piston head 1380 can engage so if one rotates the piston head one pushes the piston head front surface against the second reservoir 1370.

Note that in at least one exemplary embodiment the restoring force of the first reservoir 1310 can be such that the fluid remains in the second reservoir 1370 unless the volume of the second reservoir 1370 is decreased. Such a configuration can be used for an earplug where the portion to be inserted is collapsed into a minimal profile shape and upon insertion a user can move the structure so that the volume of the second reservoir 1370 decreases increasing the fluid in to the first reservoir, such that the first reservoir 1310 expands occluding a channel (e.g., ear canal) into which the earpiece is at least partially placed. Note that other channels can be used to convey acoustical energy across the first reservoir, for example the tube 1320 can be used to measure or emit sound to the left of the first reservoir as illustrated in FIG. 13.

FIG. 14 illustrates a non-limiting example of a moveable structure discussed with reference to FIG. 13, where the stem 1384 is attached to a tab 1400 that a user can move (e.g., push, rotate) to move the structure toward or away from the second reservoir 1370.

FIG. 15 illustrates an isolated view of the stent discussed with reference to FIG. 13. Note that for ease of manufacturing the stent can be similar to that used in an infant urology Foley catheter, which has an inflation tube 1330 and a flush tube 1320, where for an earplug the flush tube is sealed, for example by injecting a flexible curing material (e.g., Alumilite Flex 40™ casting rubber).

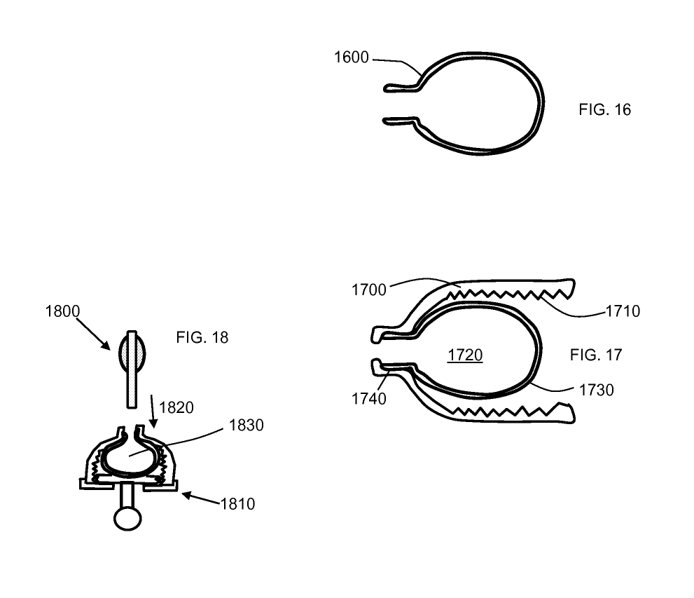

A bladder 1600 (FIG. 16) having a preformed shape (e.g., non-compliant medical balloon) or flexible shape (e.g. compliant medical balloon) can be filled with the desired fluid then attached to the stent or to the housing and sealed (FIG. 17). The bladder 1730 can be attached 1740 to housing 1700 that can also include threads 1710. The fluid filled 1830 housing 1810 (FIG. 18) (e.g., fabricated from a plastic, hard rubber) can then be attached 1820 to the stent 1800, the structure screwed into threads in the housing, and a retainer cap 1395 attached to the housing (e.g., via loctite glue) restricting the movement of the structure. Note that there can be a hole in the retainer cap 1395, having a hole diameter D_{H}, where in at least one embodiment, D_{H}, can be larger than the tab 1400 width D_{F}. Note that the bladder 1600 can be of various shape for example semi-spherical, cylindrical, and can be formed by various methods such as dip molding. Note also that an optional stop ring 1350 can be used.

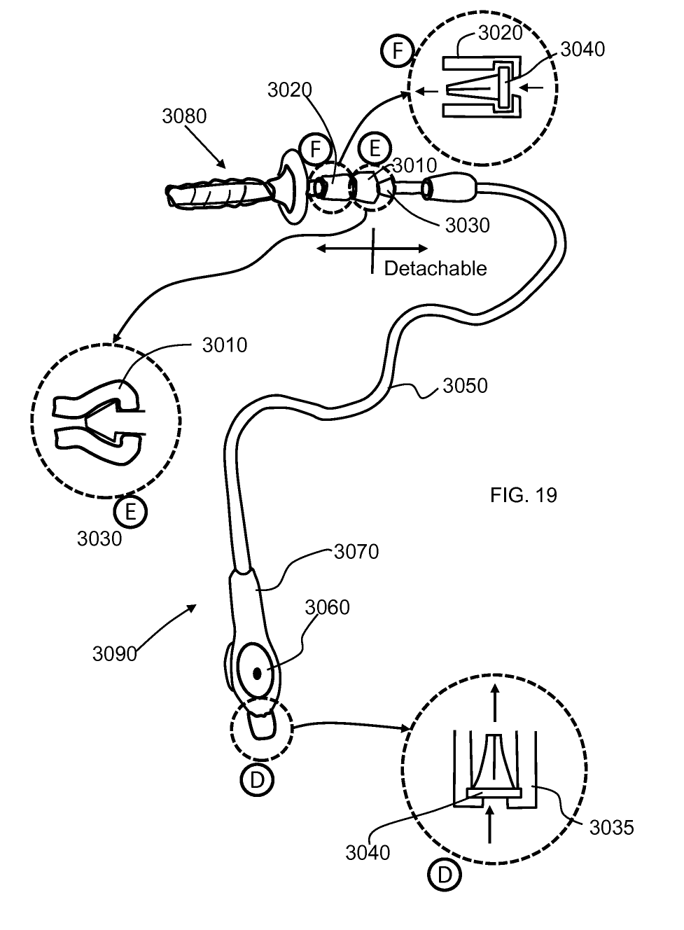

FIG. 19 illustrates a detachable earplug pumping system in accordance with at least one exemplary embodiment. The earpiece 3080 can be operatively attached via a tube 3050 to a finger pump 3090. The entire pump system (e.g., 3030, 3050, 3070, 3060, 3035) can be detachable from a pump insert port 3010. A pump seal valve 3040 in a sealing section 3020 in the earpiece 3080 generally allows one way flow and seals when the pump system is detached. The earpiece includes initially a deflated fluid reservoir which is fluid filled when the pump is actuated (e.g., finger pumped). The pump insert port 3010 allows general sealing with a detachable pumps insert interface 3030 (e.g., arrow head). The pump system can include a feed tube 3050 attached to the insert interface 3030. The feed tube can be attached to a pump body 3070 which includes a finger dimple 3060, for example fabricated from a restoring flexible material (e.g., rubber) that returns to its original shape after deformation. Thus deformation of the finger dimple 3060 forces fluid through feed tube 3050 and into the earpiece 3080. A one way valve (e.g., 3040) system 3035 feeds fluid (e.g., from the environment) into the pump body 3070 so via another deformation of the finger dimple 3060 fluid is available to be pumped into earpiece 3080.

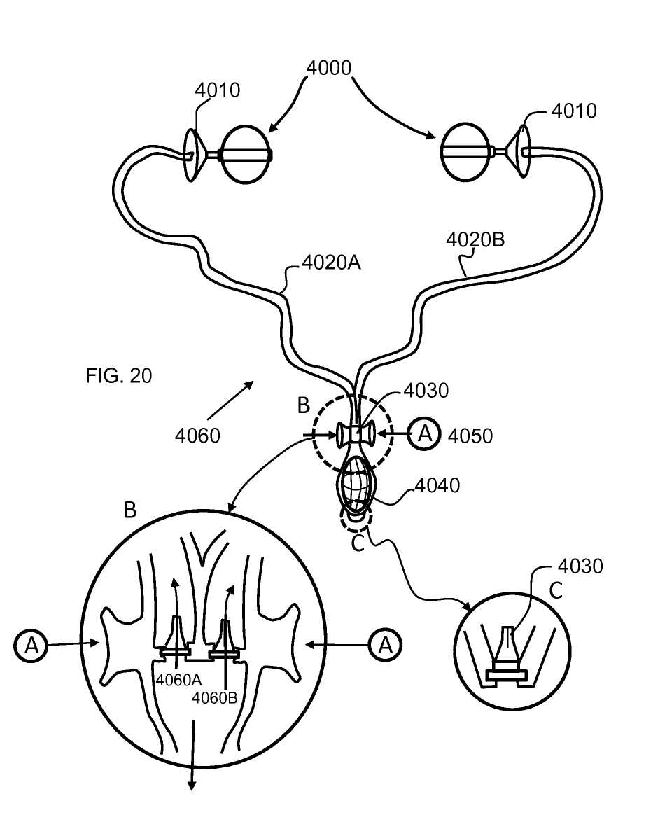

FIG. 20 illustrates a lanyard earplug system 4060 in accordance with at least one exemplary embodiment. Earpieces 4000 including optional stop flanges 4010 can be attached to a lanyard finger pump system 4060. The lanyard finger pump system can include two connected tubes 4020A and 4020B each feeding a separate earpiece 4000. The tubes 4020A and 4020B can be connected via one way valves 4030 to a squeeze release section 4050, which can be squeezed (A) to deflate the earpieces 4000. The pump section can include a finger dimple 4040 and an inlet one way valve C. The inlet one way valve C can include a one way valve 4030. The release section 4050 can include two one way valves, one 4060A associated with tube 4020A and the other 4060B associated with tube 4020B. As fluid is pushed through each one way valve 4060A and 4060B the respective earpieces 4000 inflate. An optional one way valve per tube (not shown) can be used to make sure that the maximum pressure in each tube 4020A and 4020B does not exceed a maximum value P max.

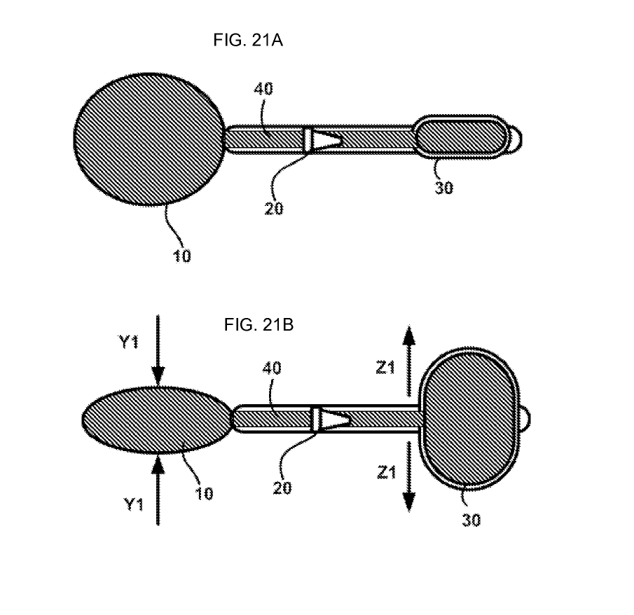

FIGS. 21A and 21B illustrate the operation of at least one exemplary embodiment. Note that materials used for construction of earplugs, hearing aids, headphones, balloons and membranes can be used to construct exemplary embodiments used as earplugs. The device includes a reservoir 10, a fluid channel 40, a valve 20 and expandable element 30. The reservoir 10 includes a medium that can be tailored to vary the acoustic spectrum as a function of frequency. The distal end (right end of FIG. 21A) is inserted into an ear canal. The user then depresses Y1 the reservoir 10, which moves fluid from the reservoir 10 through the fluid channel 40 in a single direction as provided by the one way valve 20. The fluid movement into the expandable element 30 expands (Z1) the element 30 to a desired extent. The modification of any acoustic spectrum that passes through the earplug can be tailored (acoustically shaped) by varying the medium and pressure. Various non-limiting examples of various mediums will be discussed below, but in general can include liquids, gases, mixtures, colliodal suspensions, foams, gels, and particle suspensions. For example a colloidal suspension (e.g. aphron) can be held in suspension until mixed by a user (e.g., reservoir 10 squeezed) and a chemical reaction can occur (e.g., to generate heat to warm an earplug before insertion in cold climates).

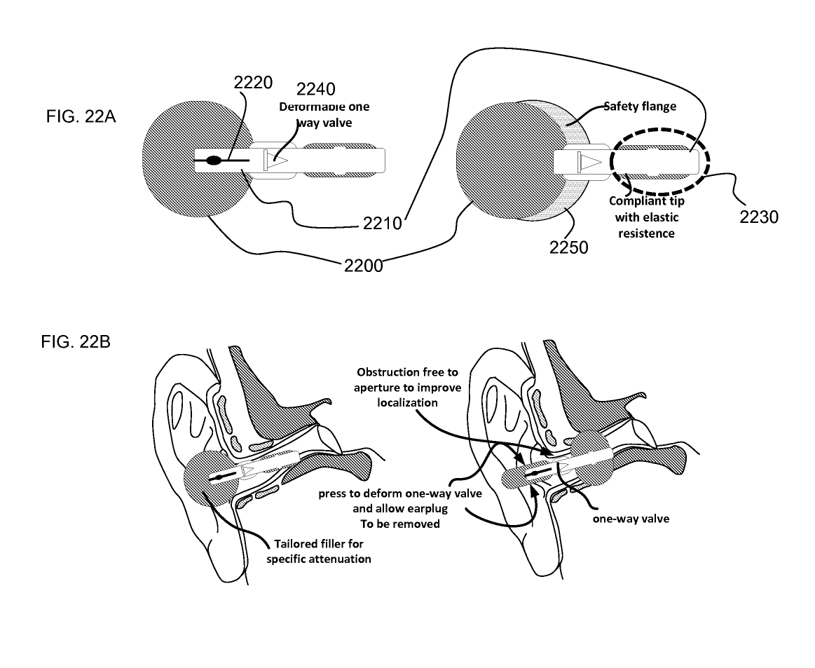

FIGS. 22A and 22B illustrate concepts of a membrane based earplug, that should fit the majority of the population (5th percentile-95th percentile), be easy to clean/maintain, be environmentally durable (e.g. durable silicone), maximize ability to detect/identify/pinpoint sounds, be lightweight, easily donned/doffed, and be compatible with currently fielded military equipment, to include helmets. The reservoir 2200 includes a medium specifically chosen, as described herein, to control the reflection and/or the transmitted attenuated acoustical spectrum. A stent 2210 with a cut 2220 (to facilitate bending), channels the medium into a flexible distal end 2230. The valve 2240 allows one way passage of the medium into the distal end expanding the distal end (see operation in FIG. 22B). To release the pressure a user can press on the stent 2210, which bends because of the cut 2220, placing pressure on the valve 2240 opening the valve deflating the distal end. Alternatively a house for a safety flange 2250 can be designed so that a user can squeeze the safety flange toward the stent to deform and open the valve 2240.

At least one example, FIGS. 23A and 23B, illustrates of an exemplary embodiment, includes an earplug 2300 with no valve, for example employs a manual push dimple/tab 2310 system having a fastener in the reservoir (e.g., Velcro™) that fastens (e.g., portion 2320 interlocking 223 with portion 2330) when pushed holding the fluid (gas, or liquid) in an inflation element until manually released (e.g., tab 2310 pulled to pull apart the Velcro™). The holding force F_{L} of the reservoir internal fastener must exceed the natural restoring force of the expanded inflation element 2370. To release the expanded distal end a user pulls the tab to overcome the internal holding force of the reservoir. Note that the earplug 2300 can include a body 2350 having various thickness, encompassing a fluid chamber 2340, connected to a channel 2360, terminating at an inflation element 2370. When the tab 2310 is pushed Z the fluid moves from the chamber 2370 into and inflating at least a portion of the inflation element 2370.

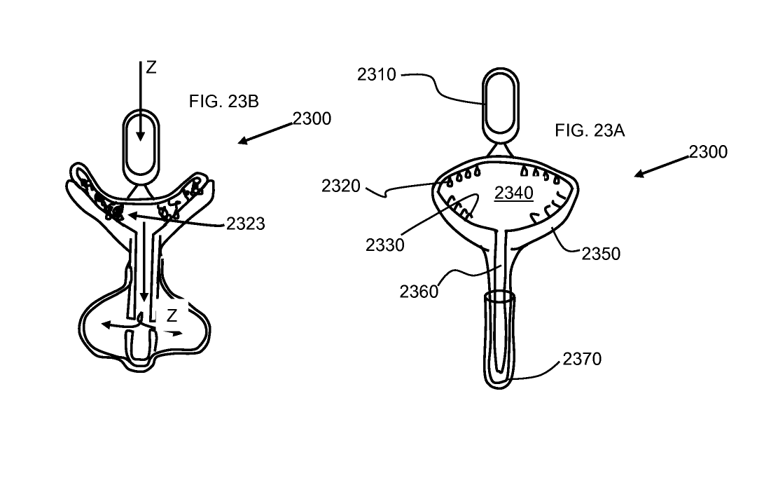

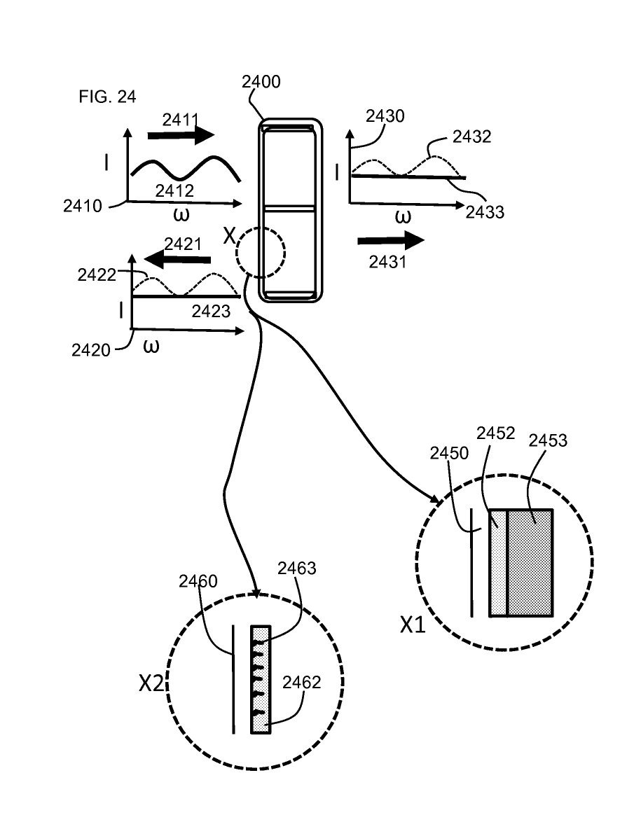

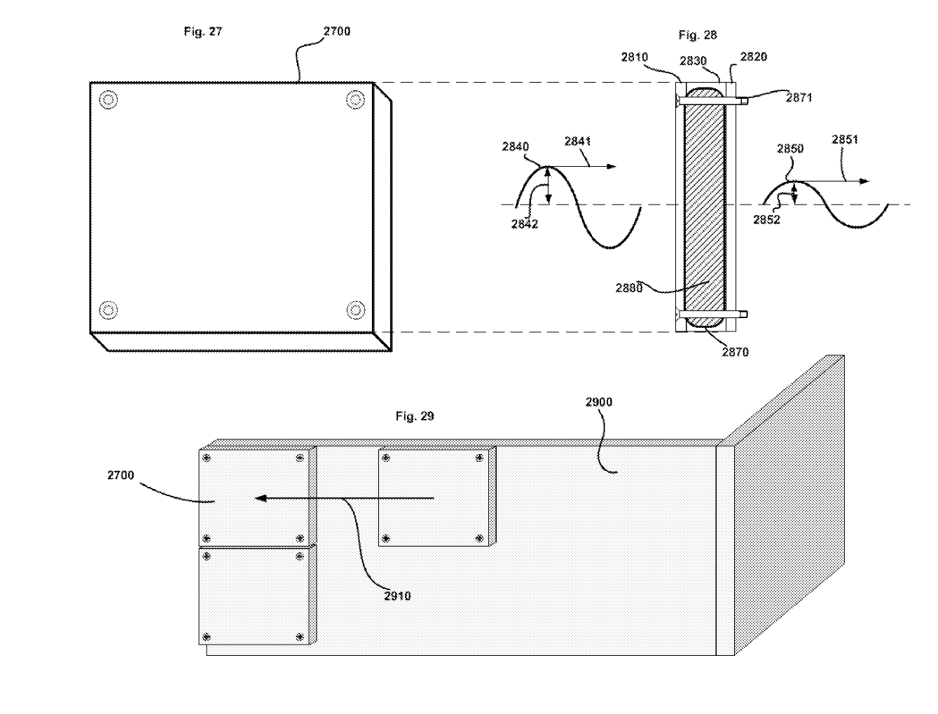

At least one further exemplary embodiment can be used as a sound panel or insert, described in more detail below with respect to FIGS. 27-31D. FIG. 24 illustrates an example of an embodiment 2400 where the membrane and/or medium can be designed to tailor the transmitted attenuated sound profile and/or the reflected sound profile (e.g., for use in concert halls). For example small filaments 2463 can be built into the membrane 2462 to absorb sound frequencies associated with the natural frequency of the filaments. The Panel 2400 can be configured such that incident wave 2411 having a spectrum 2412 incident on the panel 2400 results in a reflected wave 2421 having spectrum 2423, and a transmitted wave 2431 having a spectrum 2433, where the panel has modified the initial spectrum passing through the panel 2432 into the resultant transmitted spectrum 2433. The Panel 2400 can include several layers including a combination membrane X1 that includes a membrane 2450 under tension an absorptive layer 2452 and a medium 2453 FIG. 25A illustrates an embodiment 2500 where the membrane 2550 includes cavities 2560 that can be filled with or without (e.g., gas, liquids, suspended solids) mediums to design particular resonant frequencies 2525 associated with the cavities, affecting both the transmitted and reflected acoustical spectrum. FIG. 25B illustrates an embodiment 2600 where the membrane is composed of electroactive polymers, (e.g., Nafion™) where a voltage difference across the membrane can stiffen the membrane affecting the reflected and transmitted acoustical profiles. For example a treated Nafion™ membrane, for example as done for artificial muscle research, can be used as the membrane 2630 for a panel, where the voltage 2610 across the membrane (‘across’ with respect to exterior and interior) can be low initially (e.g., 0.25 volts). When enhanced reflection is desired the voltage can be increased (e.g., 1.5 Volts). The fabrication of artificial muscles as known by one of ordinary skill in the art is described in EP Patent Application 0924033 A2, filed 14 Dec. 1998 incorporated by reference in its entirety.

FIG. 25C illustrates an additional embodiment 2690 where a sound panel/insert in accordance with an embodiment has been inserted into an ear muff to tailor the spectrum attenuated. An additional embodiment includes the use of the sound panel/insert as an outer soft shell 2692 of the earmuff, focusing on reflecting a portion of the spectrum before attenuation. A description of the fabrication of an earmuff is described in EP Patent Application No. EP 1811932 B1, filed 16 Nov. 2005 incorporated by reference in its entirety. For example an example of a sound insert in accordance with at least one embodiment of the present invention can be incorporated into the cup shaped cap and/or as part of or in place of the pressure-equalizing means in application EP 1811932. the

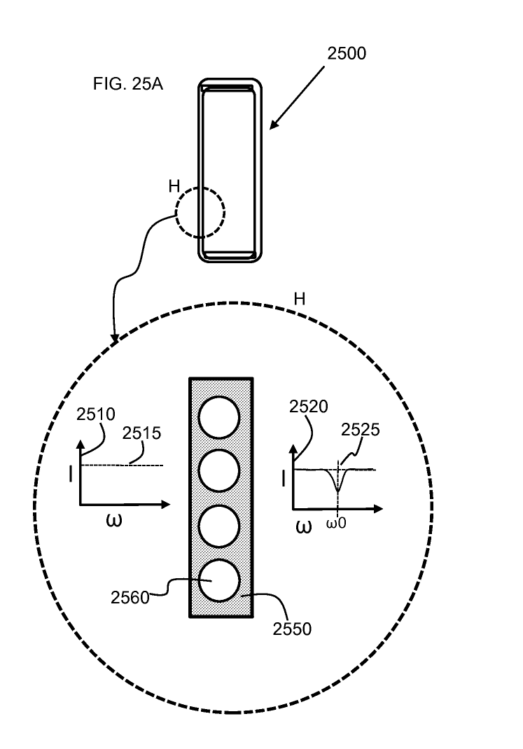

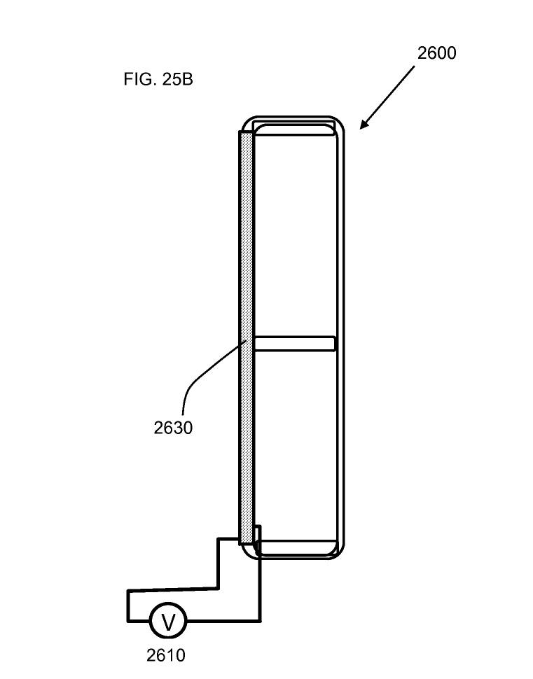

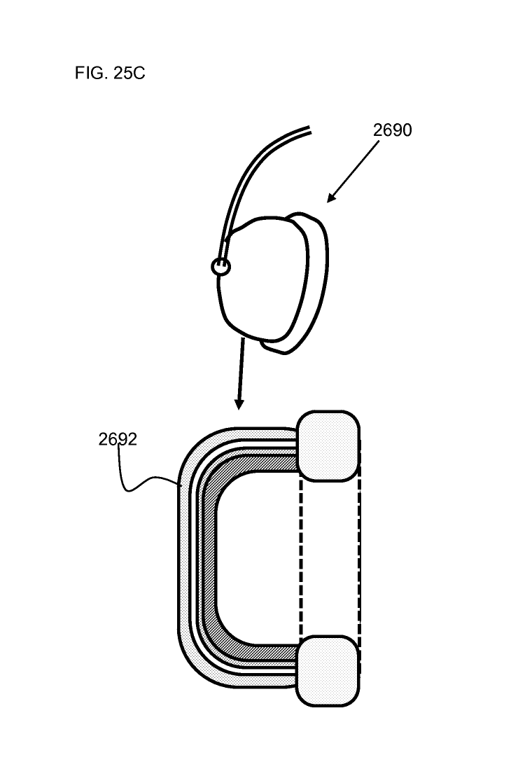

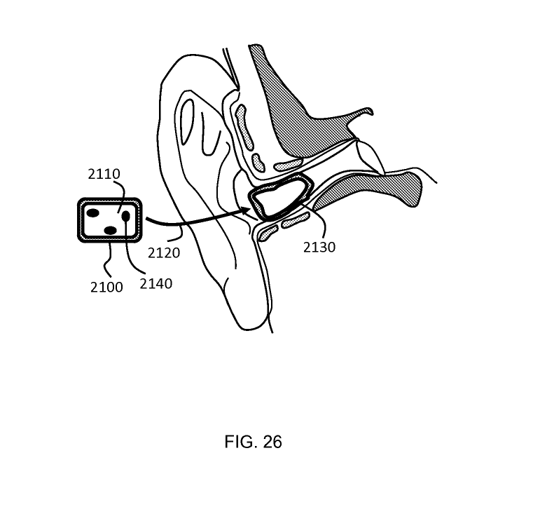

FIG. 26 illustrates at least one exemplary embodiment of an earplug (e.g., foam, polymer flange) with a hollow chamber 2100, which has a filler material (e.g., water, aphrons, water with solid particles suspended, oil with particles suspended 2140), that can be compressed and inserted 2120 into a compacted form 2130 in the ear canal. Note that while compacting the earplug the pressure of the interior can increase. The suspended particles or aphrons 2140 can be tailored with various materials tailored to the specific attenuation properties desired.

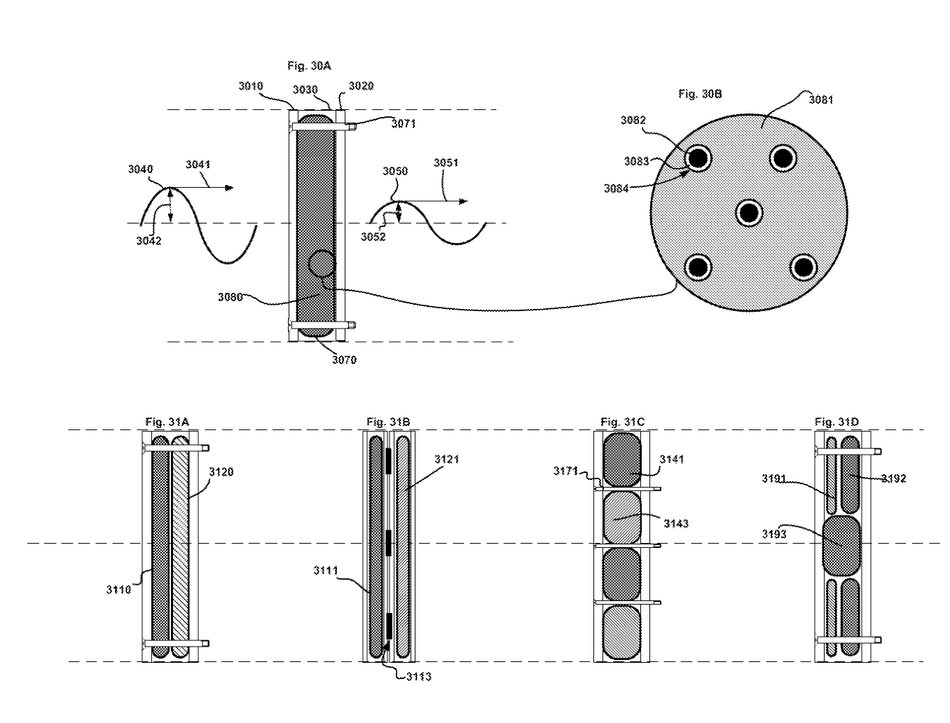

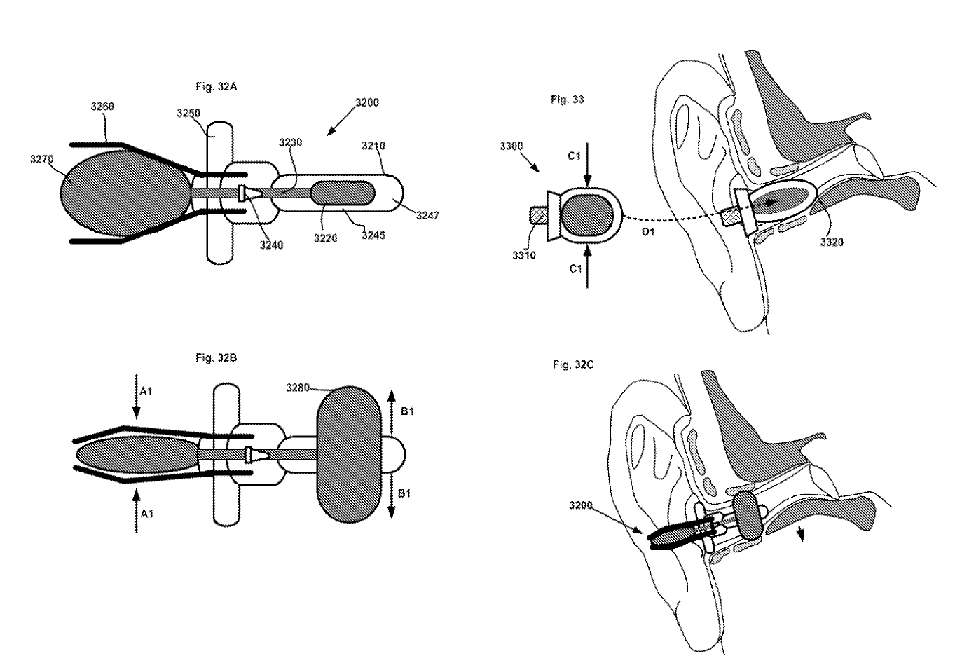

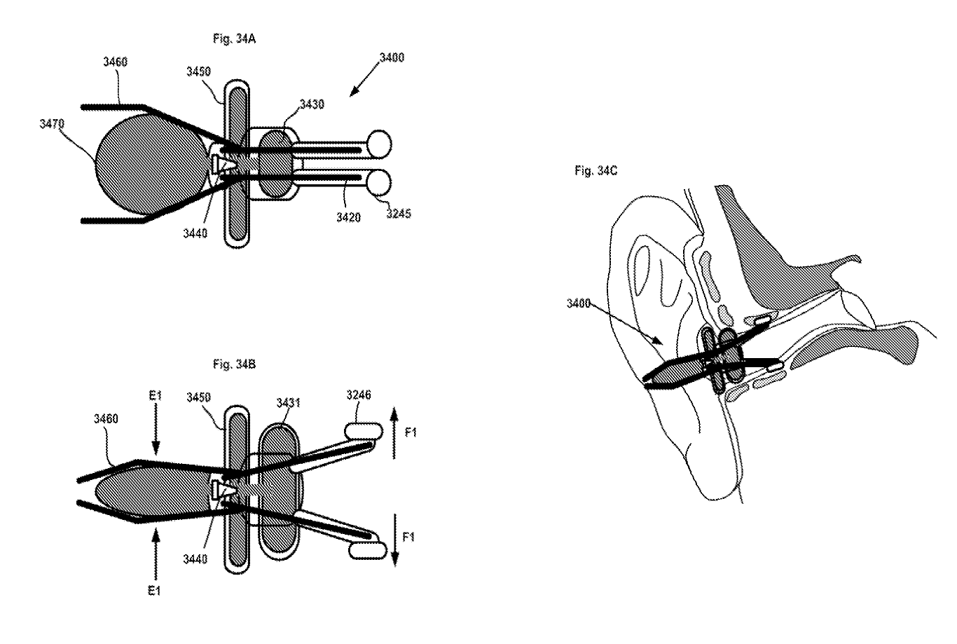

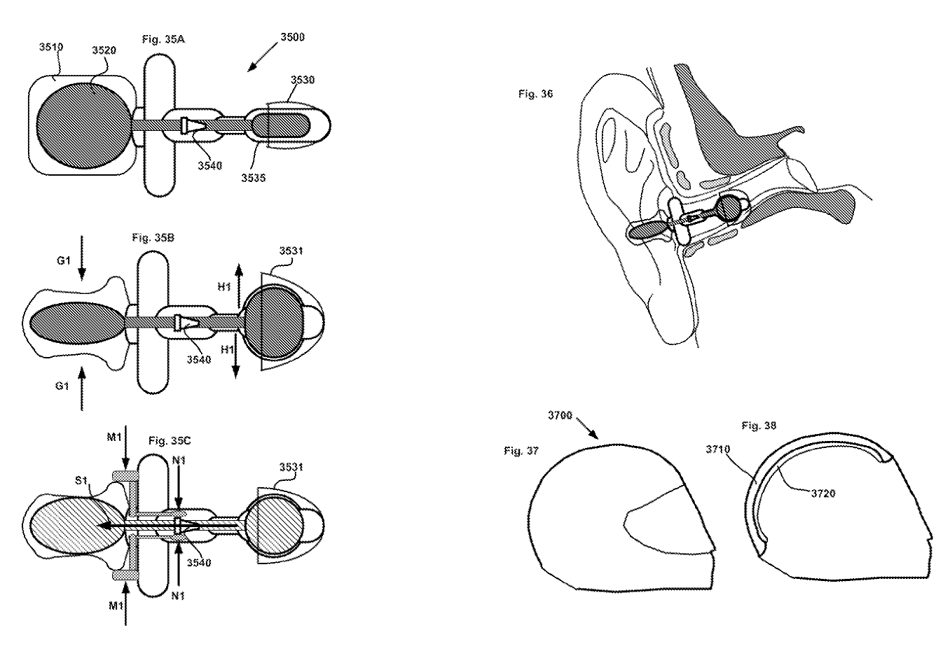

FIG. 27 illustrates an acoustic shaping panel 2700 in accordance with at least one exemplary embodiment and FIG. 28 illustrates a cross section of the panel illustrated in FIG. 27. The panel 2700 can include fastening elements 2871, or can have attachment elements on at least one side of the panel 2700 (e.g., Velcro™ attachment). Referring to FIG. 28, an incident 2841 acoustic wave 2840 (only one frequency illustrated for clarity) with amplitude 2842 passes through the panel 2700. Depending upon the desired acoustic shaping, the panel 2700 will modify different frequencies in various methods, for example reducing the amplitude (measured in Decibels or dB). The transmitted 2851 acoustic wave 2850 has a reduced amplitude 2852. The reduction amount of the incident amplitude (2852) is a function of the properties of the case (e.g. front 2810, back 2820, and rim 2830) of the panels and the properties of the medium 2880. The medium 2880 can be contained within a medium retainer container 2870 (e.g., a bladder). The medium 2880 can be inserted under various pressures to obtain various levels of amplitude reduction (e.g., attenuation).

FIG. 29 illustrates attachment of the panels of FIG. 27 on a wall 2900 in accordance with at least one exemplary embodiment. In the non-limiting example illustrated, the acoustic properties of a wall 2900 can be modified by adding multiple panels 2700 which are placed 2910 next to each other.

FIG. 30A illustrates cross section of an acoustic shaping panel in accordance with at least one exemplary embodiment. Referring to FIG. 30A, an incident 3041 acoustic wave 3040 (only one frequency illustrated for clarity) with amplitude 3042 passes through the panel. Depending upon the desired acoustic shaping, the panel will modify different frequencies in various methods, for example reducing the amplitude (measured in Decibels or dB). The transmitted 3051 acoustic wave 3050 has a reduced amplitude 3052. The medium 3080 can be contained within a medium retainer container 3070 (e.g., a bladder). FIG. 30B illustrates a closeup of the medium illustrated in FIG. 30A. In the non-limiting example illustrated in FIG. 30B the medium 3081 includes a suspension 3084, for example an aphron including a sheath 3083 and core 3082. For example the sheath 3083 could be an aqueous solution including a surfactant and a core 3082 including a mixture for example oil, or H2O+NaCl, or other mixtures.

FIGS. 31A, 31B, 31C, and 31D illustrate variations of cross sections of acoustic shaping panels in accordance with various exemplary embodiments. Panels can include various combinations of mediums to shape the acoustic properties of the panels, or a combination of individual panels. For example FIG. 31A illustrates two mediums 3110 and 3120 that can be combined to provide an overall panel property, while FIG. 31B illustrates two panels attached 3113 (e.g. via Velcro™, glue, screws, nails). Additional non-limiting examples are illustrated in FIGS. 31C and FIGS. 31D, where various combinations of mediums are combined to provide tailored acoustic shaping properties of the panels. For example FIG. 31C includes mediums 3141 and 3143 and fasteners 3171 and FIG. 31D includes multiple mediums 3191, 3192, and 3193.

FIGS. 32A, 32B, and 32C illustrate the configuration and operation of at least one exemplary embodiment of an earplug. The earplug 3200 includes a reservoir 3270, a moveable element 3260, a safety flange 3250, a valve 3240, a fluid channel 3230, a distal end reservoir 3220, and a distal end shaft 3210. The shaft 3210 can expand for example including regions of various thicknesses (e.g., a thin region 3245 and a thicker region 3247), or the shaft can have a port from the distal end reservoir to a flexible element around the distal end of the shaft 3210 which expands while the shaft remains generally constant.

FIG. 32C illustrates operation of the earplug Illustrated in FIG. 32A, where the moveable element 3260 is depressed (squeezed) for example by a user's fingers, to constrict the reservoir 3270. The constriction of reservoir 3270 forces the medium in the reservoir through the channel 3230 past the valve into the distal end reservoir 3220. The passing of the medium through the valve 3240 prevents the return of the medium into the reservoir 3270, thus once depressed the fluid remains in or near the distal end reservoir. If the shaft is flexible then the thin wall portion 3245 will expand 3280 in response B1 to the reservoir constriction A1. Note that a flexible element (not shown) can be encased around the shaft where fluid entering the distal reservoir travels via a port to the flexible element expanding the flexible element, which becomes the expandable element 3280. Note that a modification to the non-limiting example illustrated can include a second return valve that opens when a design pressure is reached, for example if one seeks to remove the earplug, when upon pulling the pressure is greater than a designed level (e.g., 400 mbar gauge pressure) then medium will flow from the distal reservoir 3220 to the reservoir 3270. FIG. 32C illustrates use of the earplug 3200 in an ear.

FIG. 33 illustrates another non-limiting example of an embodiment. The earplug 3300 is a foam plug with a reservoir and a finger tab 3310 to hold. A user squeezes C1 the foam to a smaller insertion form, which is inserted D1 into the ear canal 3320. Note that the reservoir can include a fluid foam medium, which can be compressed (for example where the gas bubbles get smaller upon compression), thus increasing the pressure of the inserted reservoir.

FIGS. 34A, 34B, and 34C illustrate the configuration and operation of at least one exemplary embodiment. The earplug 3400 includes a reservoir 3470, a moveable element 3460 with a distal end 3420, a safety flange 3450, a valve 3440, a fluid channel 3430, a distal end reservoir 3430 and at least one contact 3245. Note the safety flange 3450 can additionally include a flange reservoir. FIG. 34B illustrates the operation the earplug 3400, where the reservoir 3470 is constricted by moving E1 (squeezing) the moveable element 3460 forcing a portion of the medium through the valve 3440 into a distal end reservoir 3431 and optionally a safety flange reservoir. The medium moving into the distal reservoir 3431 allows the reservoir to remain constricted by the valve 3440 prohibiting backward flow. The contacts 3245 move as the moveable element 3460 is moved, where the contact 3245 press lightly against the walls of the ear canal securing the earplug. FIG. 34C illustrates use of the earplug 3400 in an ear.

FIGS. 35A, 35B, 35C, and 36 illustrate the configuration and operation of at least one exemplary embodiment. FIG. 35A illustrates a non-limiting example of an earplug embodiment 3500, including a deformable casing 3510 (e.g. foam), encircling a reservoir 3520, a valve 3540, a flexible distal end 3535, and optionally a flange 3530. FIG. 35B illustrates the operation of the earplug 3500, where depression G1 of the deformable casing 3510 constricts the reservoir 3520 forcing a portion of the medium past the valve into the flexible distal end 3535 (e.g., a balloon on a shaft, a flexible shaft with varying thickness) expanding H1 the flexible distal end 3535. The expansion of the flexible distal end 3535 can expand the flange 3531. In at least one embodiment a release mechanism can be included for a user to squeeze open a flexible valve, allowing passage of the medium from the flexible distal end 3535 back to the reservoir 3520. For example FIG. 35C illustrates an incorporated release mechanism that when pressed M1, effectively presses N1 on the flexible valve opening the valve for backflow. FIG. 36 illustrates use of the earplug 3500 in an ear.

Although considerable discussion has been included with respect to use in earplugs, additional embodiments of the invention can be used in other systems and devices that can benefit from controlling the acoustic spectrum passing through the device. For example, helmets, flexible wrap that is wrapped around devices for acoustic isolation, tool handles (e.g., jackhammers), around the hull of ships to mitigate acoustic loss, and other uses one of ordinary skill in the relevant art would know. For example FIG. 37 and FIG. 38 illustrates an embodiment used in a helmet 3700 (e.g. for use on aircraft carriers or other noisy environments) where several liners 3710 and 3720 (although a single liner can be used), where the liners (3710 and/or 3720) each can include different fluid mediums to shape the acoustic profiles entering the helmet 3700.

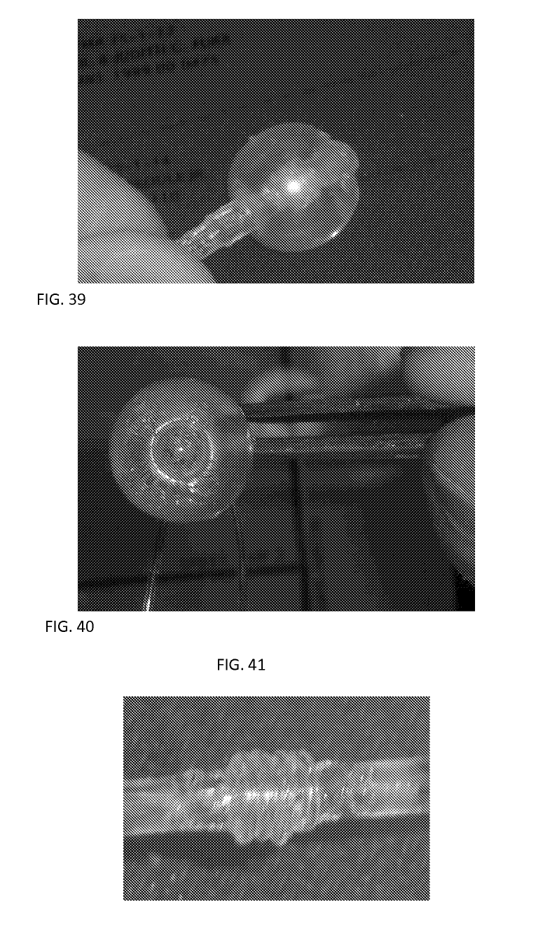

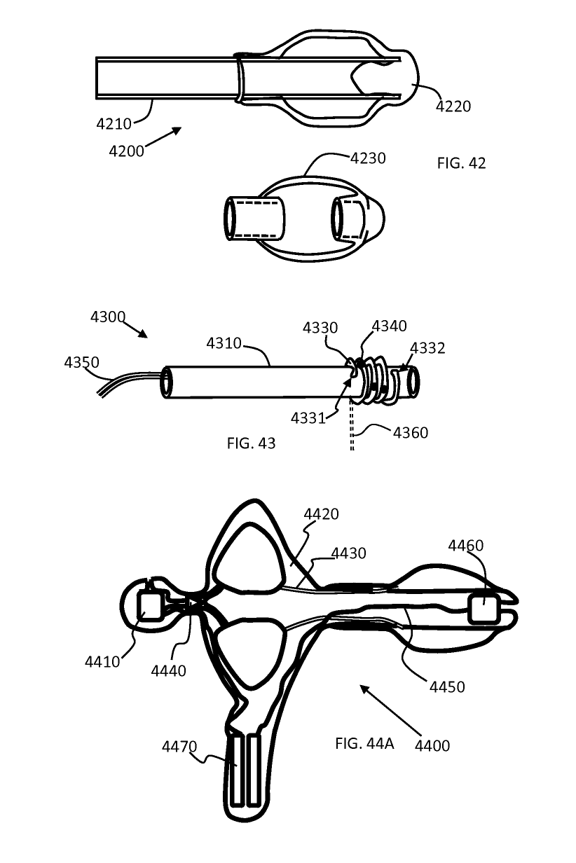

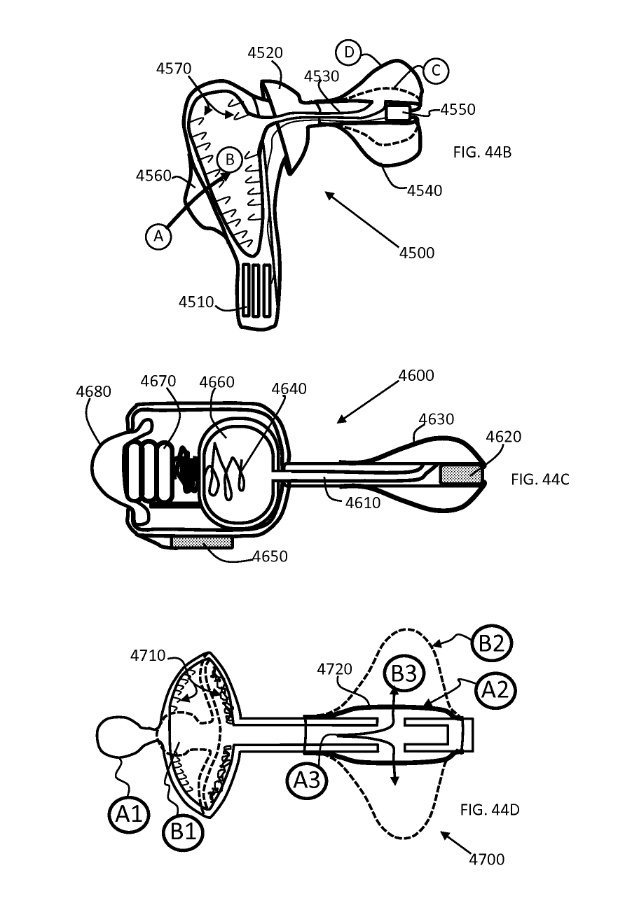

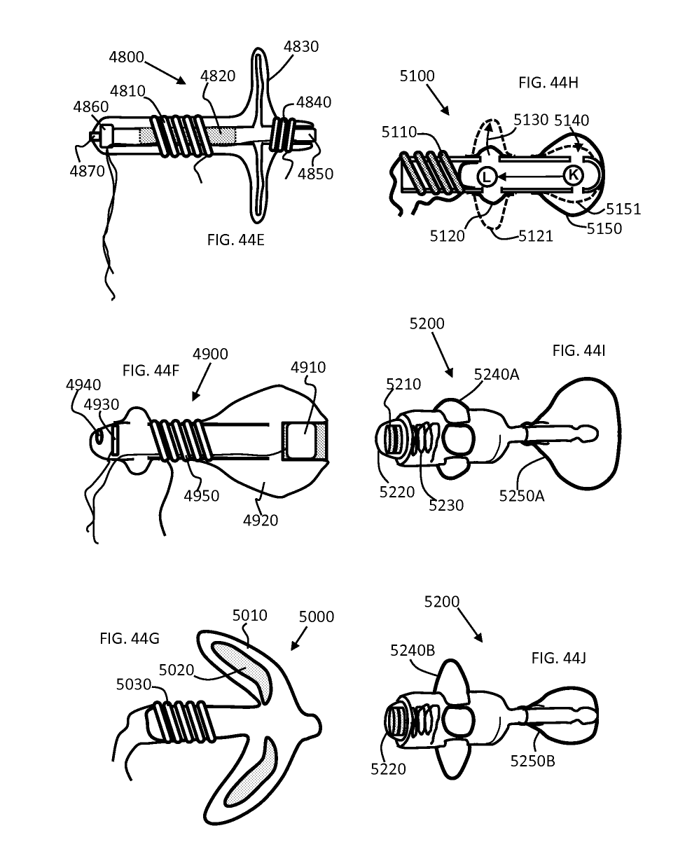

FIGS. 39-40 illustrates various flexible distal ends developed by Innovation Labs and Dr. Keady, while FIG. 41 illustrates a novel distal end spiral feed system which enhances uniform expansion about a stent. FIGS. 42-44J illustrates multiple examples of embodiments for earplugs, hearing aids, and earpieces. FIG. 42 illustrates an earplug 4200 including a stent 4210 and an inflation element 4220. The inflation element can be formed as a single unit 4230. FIG. 43 illustrates the system of FIG. 40. An inflation tube 4350 can be placed through a hole 4331, then an end 4360 wrapped 4330 around the stent 4310, with holes 4340 in the inflation tube. When the winding in completed the end of the inflation tube 4350 is inserted through a second hole 4332. FIG. 44A illustrates an earplug/hearing aid 4400 having an ambient microphone 4410, a body 4420 that serves also as a depth control flange, inflation tubes 4430 (feed tubes) a one way vent 4440 (e.g. valve), receiver/microphone 4460, and wires 4450. FIG. 44B illustrates an embodiment of an earplug/hearing aid 4500, including inflation tubes 4530, depth control flange 4520, inflation element 4540, tab 4560, interlocking mechanism 4570, batteries 4510, where a user that presses inward, A to B, forces fluid into the inflation element 4540 expanding the inflation element 4540 from C to D. FIG. 44C illustrates an embodiment of an earplug/hearing aid 4600, including an inflation tube 4610, a button 4680 pressing the batteries 4670, where a user can press the button 4680 engaging the battery 4670 to supply voltage to electrodes 4640. Where the electrodes 4640 are embedded in a medium 4660 (e.g., water) to turn the medium into gas (e.g., electrolysis), where the gas and fluid have a increased pressure that expands the inflation element 4630. The earplug/hearing aid 4600 can include an ambient microphone 4650, and an internal receiver/microphone 4620. FIG. 44D illustrates an earplug 4700 including an interlocking mechanism 4710 where when a user moves a tab from position A1 to B1 moves fluid in a reservoir, from A3 to B3, into an inflation element 4720 expanding the inflation element from A2 to B2. FIGS. 44E, 44F, 44G and 44H illustrate ferrofluid and/or electro fluid earplug/hearing aid systems. For example FIGS. 44E and 44F include ambient microphones 4860, 4930, each having a microphone ports 4870 and 4940. FIGS. 44E and 44F additionally include coils 4810, 4950, which can be used to change local magnetic fields, ferrofluid 4820, 4920, and in the case of earplug/hearing aid 4800 an opposing coil 4840. The ferrofluid can react to the magnetic fields moving into and way from the inflation elements 4830, expanding them. FIG. 44G illustrates a ferrofluid system 5000 with ferrofluid 5010 in isolated chambers in a flange 5010 where a coil 5030 changes the local magnetic field collapsing or releasing the flange 5010. The earplug system 5100 illustrated in FIG. 44H includes a restoring membrane 5120 that when expanded 5121 exerts a restoring force attempting to impose the attraction of the ferrofluid responding to an increased magnetic field (e.g., moving from K to L). When the magnetic field is released (current to minimal) the restoring membrane forces the ferrofluid into the inflation element expanding it from 5151 to 5150. FIGS. 44I and 44J illustrate the same system using a push button 5220 to engage the battery 5210 with the magnetic coil 5230 increasing the current and applying a magnetic field which attracts the ferrofluid from the tip to the restoring membrane region expanding the restoring membrane 5240B.

Additional exemplary embodiments use a field responsive fluids (e.g., Electric and Magnetic Fluid Technology: Any device portion that includes ferrofluids, magnetorheological fluids, and Electro-rheological fluids/electric field responsive fluids. For example one exemplary embodiment uses a magnetic generator (e.g., coil) to control FerroFluid in an earpiece to move from one point of the earpiece to another, and/or to change the attenuation characteristics of the earpiece. At least one exemplary embodiment uses an ER fluid to change the attenuation properties via the application of an electric field. For example for an earpiece if the insertion depth control flange contains an ER fluid the viscosity of the fluid can be changed by applying an electric field across the flange changing the characteristics of the flange.

At least one exemplary embodiment also use a combination ER and FF fluid by mixing them so that a magnetic field can be used to move the fluid while an electric field can be used to gellify the fluid.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions of the relevant exemplary embodiments. For example, if words such as “orthogonal”, “perpendicular” are used, the intended meaning is “substantially orthogonal” and “substantially perpendicular” respectively. Additionally, although specific numbers may be quoted in the claims, it is intended that a number close to the one stated is also within the intended scope, i.e. any stated number (e.g., 20 mils) should be interpreted to be “about” the value of the stated number (e.g., about 20 mils).

Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

What is claimed is:

1. An earpiece comprising:
a fluid reservoir, including an internal fastening system;
a channel; and
a flexible distal end, where the fluid reservoir is operatively connected to the distal end by the channel, where the reservoir is configured to be expanded when the internal fastening system is not engaged, and where the reservoir is configured to push fluid through the channel into the distal end when one side of the reservoir is pressed, and where the reservoir is configured so that the when the one side is pressed the internal fastening system engages facilitating the reservoir staying in a pressed state.

2. The earpiece according to claim 1, where the fluid is at least one of a gas or a liquid.

3. The earpiece according to claim 2, where the fluid is at least one of water or a mixture of water.

4. The earpiece according to claim 2, where the internal fastening system is Velcro™.

5. The earpiece according to claim 5, further comprising:
a housing, where the second reservoir is within the housing, where a portion of the structure is within the housing.

8. The earpiece according to claim 4, where the pressed state is released by a user pulling on the reservoir in a direction opposite to the direction the reservoir was initially pressed.

9. An earpiece comprising:
a fluid reservoir;
a pump insert port, where the pump insert port is configured to attach to a detachable pump system so that when the pump system is activated the fluid reservoir fills with fluid; and
a pump seal valve, where the pump seal valve is configured to close when the pump system is detached from the pump insert port.

10. An earpiece system comprising:
a first fluid reservoir;
a second fluid reservoir;
a lanyard operatively connecting the first fluid reservoir and the second fluid reservoir; and
a pump operatively connected to the lanyard, where the pump modifies fluid flow in the first and second fluid reservoirs.
